(19) United States
(12) Patent Application Publication
(10) Pub. No.: US 2007/0207230 A1
(43) Pub. Date: Sep. 6, 2007

(54) EXTRACT OF NELUMBINIS SEMEN FOR THE TREATMENT OF DEPRESSION, MEDICINAL COMPOSITE AND HEALTH FOODS INCLUDING THE EXTRACT OF NELUMBINIS SEMEN

(75) Inventors: Hyun-Su Bae, Seoul (KR); Moon-Kyu Kang, Namyangju-si (KR); Jung-Wan Oh, Seongnam-si (KR); Chong-Woon Cho, Gyeryong-si (KR); Chang-Sook Kim, Ansan-si (KR); Hwa-Jin Lee, Seoul (KR); In-Sop Shim, Seongnam-si (KR); Choon-Gon Jang, Suwon-si (KR); Hyun Choi, Seoul (KR); Eun-Jung Ko, Seoul (KR); Chang-Jun An, Seoul (KR); Hyun-Taek Kim, Seoul (KR); Moo-Chang Hong, Seoul (KR); Min-Kyu Shin, Seoul (KR)

Correspondence Address: SHERIDAN ROSS PC, 1560 BROADWAY, SUITE 1200, DENVER, CO, 80202, US

(73) Assignee: PURIMED CO., LTD., Seoul (KR)

(21) Appl. No.: 10/599,546

(22) PCT Filed: Mar. 31, 2005
(86) PCT No.: PCT/KR05/00932

§ 371 Date: Sep. 29, 2006

(30) Foreign Application Priority Data
Mar. 31, 2004 (KR) 10-2004-0022413

(51) Int. Cl. A61K 36/185

(52) U.S. Cl. 424/776

(57) ABSTRACT

Disclosed are an extract of Nelumbinis Semen (Nelumbo nucifera) having an antidepressive effect, and a pharmaceutical composition and a health food for treating depression, comprising the extract, The Nelumbinis Semen extract is prepared by extracting Nelumbinis Semen with hot water. Animal behavioral and biochemical studies revealed that the Nelumbinis Semen extract has very strong antidepressive activity and is safe to the body while reducing the side effects of conventional antidepressants. Thus, the Nelumbinis Semen extract is applicable to a composition and a health food for treating depression.

### TECHNICAL FIELD

The present invention relates to an extract of Nelumbinis Semen (Nelumbo nucifera) having a therapeutic effect on depression, and a pharmaceutical composition and a health food comprising the extract. More particularly, the present invention relates to a Nelumbinis Semen extract prepared by extracting Nelumbinis Semen with hot water, and a pharmaceutical composition and a health food for treating depression, which comprise the Nelumbinis Semen extract as an effective component.

### BACKGROUND ART

Mental damage occurring in the complicated modern society is, contrary to in the past, mostly caused by weak but prolonged and repeated stress from usual activities rather than large psychological impact or stimuli. Such stress is difficult to be recognized by patients and easily overlooked during hospital visits by patients, and thus accumulates, causing individuals to suffer from depression.

Depression is an emotional pathological phenomenon occurring regardless of objective situations. Emotional symptoms of depression include depressed behavior during all activities, anhedonia (loss of interest or pleasure), diminished mental capacity, pessimism, poor self-esteem, and suicidal thoughts that occasionally lead to suicide attempts. Physical symptoms of depression include decreased appetite, insomnia, constipation, diminished sexual desire, reduced immune functions, and patients' susceptibility to diseases due to the reduced immune function.

There has been so far no theory that perfectly explains the mechanism causing depression and the action mechanism of antidepressants for treating depression. However, for many years, the prevailing hypothesis is that depression is caused by an absolute or relative deficiency of monoamine neurotransmitters in synapses of the central nervous system, such as serotonin, norepinephrin and dopamine. In this regard, all antidepressants have pharmaceutical action to increase concentrations of neurotransmitters in central serotonin or noradrenaline synapses.

Antidepressants are divided into three major types according to the mechanism involving increasing the neurotransmitter levels: tricyclic antidepressants (TCA) monoamine oxidase inhibitors (MAOI) and selective serotonin reuptake inhibitors (SSRI).

Monoamine oxidase inhibitors, such as phenelzine developed a relatively long time ago, have a severe adverse effect of inducing heart diseases, and thus, have not been widely used recently. Tricyclic antidepressants such as imipramine also have anti-cholinergic, sedative, and other side effects related to the cardiovascular system. Thus, recent research focuses on the development of therapeutic agents against depression using selective serotonin (5-HT) reuptake inhibitors (hereinafter, referred to simply as “SSRI”) as antidepressants with fewer side effects. Representative examples include fluoxetine (brand name: Prozac), paroxetine (brand name: Seroxate), and sertraline (brand name: Zoloft), which are widely approved due to their clinical efficacy.

However, the aforementioned drugs also have side effects such as whole-body fatigue, sexual dysfunction and insomnia. Administration of antidepressants was reported to typically activate a serotonin receptor by increasing serotonin levels, leading to an activation of PKA that is downstream of the serotonin receptor and eventually increases in protein levels of CREB, and brain-derived neurotrophic factor (BDNF) and its receptor trkB. These increased protein levels are considered to indicate effective actions of antidepressants in molecular levels, and the increased BDNF levels or increased serotonin receptor activity recover the damaged memory of depressed patients by inducing nerve cell regeneration particularly in the hippocampus, that is, neuronal regeneration (J. of Psychosomatic Research 53, 687-697 (2002)). In addition, the administration of antidepressants restores to normal levels decreased concentrations of cortisol and IL-2 and decreased cell numbers of WBC and lymphocytes, all of which are representative responses of individuals with depression, thereby providing a normal immune system (Ann N Y Acad Sci. 917, 478-487 (2000)). These effects may be another therapeutic effect of antidepressants.

Recently, in the Western countries, mecidinal herbal extracts have been recognized for their therapeutic effects and studied. With regard to depression, extracts of Hypericum perforatum (known also as St. John's wort) have been studied (Neuropharmacology, 1999, 21(2), 247-257; Cochrane Database Syst Rev, 2000, (2), CD000448; Drugs Aging, 2000, 16(3), 189-197).

According to a report that compared a Hypericum perforatum extract with imipramine for therapeutic efficacy on depression, the H. perforatum extract has similar efficacy to imipramine in treating depression and has fewer side effects (BMJ, 2000, 321, 536-539). Also, the H. perforatum extract has the potential to inhibit the activities of human cytochrom P450 enzymes (J Pharmacol Exp Ther, 2000, 294(1), 88-95).

The H. perforatum extract contains a large number of structurally different compounds that directly or indirectly affect the central nervous system (CNS). That is, the H. perforatum extract contains bioactive compounds, such as hypericin and hyperforin, and dimeric flavors, which are known to have antidepressive and apprehension-removing effects in animals and humans.

The action mechanisms of the constituents of H. perforatum are as follows. Hypericin is proved to have the antidepressive effect in the presence of dimeric pro-cyanidines contained in the H. perforatum extract (Regensburg, Germany, V. Butterwecke et.al., 45th Annual Congress of the Society for Medicinal Plant Research, 1997, Abstract No. 011). Hyperforin increases 5-HT (serotonin) levels in the hypothalamus and hippocampus, indicating that the antidepressive effect of hyperforin is associated with the serotonergic system (J Pharm Pharmacol, 2001, 53(5), 583-600; Pharmacopsychiatry, 2000, 33(2), 60-65). However, about 20% of depressed patients are not treated with conventional antidepressants, and recently developed antidepressants such as SSRI have fewer side effects than other antidepressants, but they are still not negligible.

On the other hand, various depression animal models have been tried in the development process of antidepressants for treating depression. Strong stimuli such as intense foot-shock, cold water immersion and 48 h food/water deprivation were initially preferred, but, recently, preferred methods are to use weak repetitive stresses better capable of mimicking usual activities of modern people experiencing weak prolonged chronic stresses (Psychopharmacology, 1984, 83, 1-16). Among the recent methods, a chronic mild stress (hereinafter, referred to simply as “CMS”) model, suggested by Willner et al., has been approved as an excellent animal model of depression having reliability and validity (Neuroscience and Biobehavial Review, 1981, 5, 231-246; TIPS, 1991, 12, 131-136).

“Mildly stressed rats” means that, when CMS-induced behavioral changes are observed for a prolonged administration period of weeks, the behavioral changes do not occur habitually, or habitual changes occur within a constant limitation (Psychopharmacology, 1997, 134, 319-320). In general experiments, a variety of chronic weak stressors, such as overnight illumination, periods of food and/or water deprivation, cage tilt and change of cage mate, are used (Psychopharmacology, 1997, 134, 319-320). Repeated exposure of white rats to such stressors results in a significant decrease in consumption of a sucrose solution, which is comparable to anhedonia, a representative symptom of depression of white rats. Upon no appropriate treatment, such decrease in consumption of a sucrose solution is known to last for several weeks after withdrawal of a CMS procedure. Many antidepressants have been approved that they have effects of recovering the reduced sucrose intake induced by the CMS procedure to an original level (Psychopharmacology, 1992, 109, 433-438).

On the other hand, Nelumbinis Semen is the skinned ripe seed of lotus (Nelumbo nucifera), which has a green core. Nelumbinis Semen has no smell and a sweet, fresh and slightly astringent taste.

Nelumbinis Semen contains a large quantity of starch and raffinose sugar, and is known to have the therapeutic effects of strengthening the spleen and stomach, alleviating insomnia, whitening the skin, relieving inflammation and healing wounds in the skin. However, to date, there is no report of its ability to alleviate depression symptoms.

### DISCLOSURE OF THE INVENTION

Technical Problem

It is therefore an object of the present invention to provide a Nelumbinis Semen extract having antidepressive activity, which is prepared by hot water extraction, and a pharmaceutical composition and a health food comprising the Nelumbinis Semen extract as an effective component.

Technical Solution

In order to accomplish the above object, the present invention provides a Nelumbinis Semen extract which is prepared by extracting Nelumbinis Semen with hot water, concentrating the extract, and drying the concentrate, and a pharmaceutical composition and a health food for treating depression, which comprise the Nelumbinis Semen extract as an effective component.

Based on the fact that Nelumbinis Semen is used as a Chinese traditional herbal medicine, intensive and thorough animal behavioral research into the therapeutic effects of an extract of Nelumbinis Semen, conducted by the present inventors, resulted in the finding that the Nelumbinis Semen extract is superior in treating depression to conventional antidepressants, Hypericum perforatum extract and fluoxetine (brand name: Prozac), which is one of SSRI, the recently most widely used type of drugs to treat depression, thereby providing a pharmaceutical composition for treating depression comprising the Nelumbinis Semen extract of the present invention minimizing the side effects of the conventional antidepressants.

In addition, the molecular biological and biochemical research revealed the mechanism of the antidepressive action of the Nelumbinis Semen extract of the present invention, and resulted in the finding that the Nelumbinis Semen extract has another effect of normalizing immune functions, thereby providing a pharmaceutical composition for treating depression comprising the Nelumbinis Semen extract of the present invention.

Further, the animal behavioral research resulted in the finding that the Nelumbinis Semen extract of the present invention does not have the side effects that are observed upon application of conventional antidepressants.

Thus, in one aspect, the present invention provides a Nelumbinis Semen extract having antidepressive activity.

In another aspect, the present invention provides a pharmaceutical composition for treating depression, comprising the Nelumbinis Semen extract as an effective component.

In a further aspect, the present invention provides a health food for treating depression, comprising the Nelumbinis Semen extract as an effective component.

Advantageous Effects

The Nelumbinis Semen extract of the present invention, which is prepared by hot water extraction, has very strong antidepressive activity. Since Nelumbinis Semen, as the raw material of the Nelumbinis Semen extract, is a natural raw material used in Chinese medicine that is not harmful to the body and is absorbed well by the body when used as a pharmaceutical composition for treating depression, the Nelumbinis Semen extract is very useful for treating and preventing depression and various related diseases.

### BRIEF DESCRIPTION OF THE DRAWINGS

FIG. 1 is a graph showing the immobility time of white rats administered with a Nelumbinis Semen extract of the present invention for one day in a forced swim test, wherein a Nelumbinis Semen treatment group is compared for immobility time with a control group, a fluoxetine treatment group and a Hypericum perforatum extract treatment group;

FIG. 2 is a graph showing the immobility time of white rats administered with a Nelumbinis Semen extract of the present invention for six days in a forced swim test, wherein a Nelumbinis Semen treatment group is compared for immobility time with a control group, a fluoxetine treatment group and a Hypericum perforatum extract treatment group;

FIG. 3 graphically shows the sucrose intake of white rats administered with a Nelumbinis Semen extract of the present invention during CMS exposure, wherein a Nelumbinis Semen treatment group is compared for sucrose intake with a normal group, a control group, a fluoxetine treatment group and a Hypericum perforatum extract treatment group;

FIG. 4 graphically shows the body weight of white rats administered with a Nelumbinis Semen extract of the present invention during CMS exposure, wherein a Nelumbinis Semen treatment group is compared for body weight with a normal group, a control group, a fluoxetine treatment group and a Hypericum perforatum extract treatment group;

FIG. 5 is a graph showing the visit counts of white rats administered with a Nelumbinis Semen extract of the present invention in an open field, wherein a Nelumbinis Semen treatment group is compared for visit counts with a normal group, a control group, a fluoxetine treatment group and a Hypericum perforatum extract treatment group;

FIG. 6 is a graph showing the start latency of white rats administered with a Nelumbinis Semen extract of the present invention in an open field, wherein a Nelumbinis Semen treatment group is compared for start latency with a normal group, a control group, a fluoxetine treatment group and a Hypericum perforatum extract treatment group;

FIG. 7 is a graph showing the rearing frequency of white rats administered with a Nelumbinis Semen extract of the present invention in an open field, wherein a Nelumbinis Semen treatment group is compared for rearing frequency with a normal group, a control group, a fluoxetine treatment group and Hypericum perforatum extract treatment group;

FIG. 8 is a graph showing the grooming time of white rats administered with a Nelumbinis Semen extract of the present invention in an open field, wherein a Nelumbinis Semen treatment group is compared for grooming time with a normal group, a control group, a fluoxetine treatment group and a Hypericum perforatum extract treatment group;

FIG. 9 is a graph showing the number of ejaculations of white rats administered with a Nelumbinis Semen extract of the present invention, wherein a Nelumbinis Semen treatment group is compared for the number of ejaculations with a normal group, a control group, a fluoxetine treatment group and a Hypericum perforatum extract treatment group;

FIG. 10 is a graph showing the post-ejaculation interval (PEI) of white rats administered with a Nelumbinis Semen extract of the present invention, wherein a Nelumbinis Semen treatment group is compared for PEI with a normal group, a control group, a fluoxetine treatment group and a Hypericum perforatum extract treatment group;

FIG. 11 is a graph showing the release of a neurotransmitter in white rats administered with a Nelumbinis Semen extract of the present invention, wherein a Nelumbinis Semen treatment group is compared for neurotransmitter release with a normal group, a fluoxetine treatment group and a Hypericum perforatum extract treatment group;

FIG. 12 is a graph showing the release of a neurotransmitter in white rats administered with a Nelumbinis Semen extract of the present invention during CMS exposure, wherein a Nelumbinis Semen treatment group is compared for neurotransmitter release with a normal group, a control group, a fluoxetine treatment group and a Hypericum perforatum extract treatment group;

FIG. 13 autoradiographically shows the results of a receptor binding assay for white rats administered with a Nelumbinis Semen extract of the present invention, wherein a Nelumbinis Semen treatment group is compared with a normal group, a control group, a fluoxetine treatment group and a Hypericum perforatum extract treatment group;

FIG. 14 autoradiographically shows the results of a receptor binding assay in the CA2 region of the hippocampus of white rats administered with a Nelumbinis Semen extract of the present invention, wherein a Nelumbinis Semen treatment group is compared with a normal group, a control group, a fluoxetine treatment group and a Hypericum perforatum extract treatment group;

FIG. 15 autoradiographically shows the results of a receptor binding assay in the CA3 region of the hippocampus of white rats administered with a Nelumbinis Semen extract of the present invention, wherein a Nelumbinis Semen treatment group is compared with a normal group, a control group, a fluoxetine treatment group and a Hypericum perforatum extract treatment group;

FIG. 16 autoradiographically shows the results of a receptor binding assay in layer I-II of the cerebral frontal cortex of white rats administered with a Nelumbinis Semen extract of the present invention, wherein a Nelumbinis Semen treatment group is compared with a normal group, a control group, a fluoxetine treatment group and a Hypericum perforatum extract treatment group;

FIG. 17 autoradiographically shows the results of a receptor binding assay in the hypothalamus of white rats administered with a Nelumbinis Semen extract of the present invention, wherein a Nelumbinis Semen treatment group is compared with a normal group, a control group, a fluoxetine treatment group and a Hypericum perforatum extract treatment group;

FIG. 18 is a photograph showing the increased or decreased proteins in white rats administered with a Nelumbinis Semen extract of the present invention, in comparison with a normal group, a control group and a fluoxetine treatment group; and

FIG. 19 is a table describing the intensity of selected protein spots, which is compared among a Nelumbinis Semen treatment group, a normal group, a control group and a fluoxetine treatment group.

### BEST MODE FOR CARRYING OUT THE INVENTION

Hereinafter, the present invention will be described in detail.

The present invention provides a Nelumbinis Semen extract having antidepressive activity, which is prepared by extracting Nelumbinis Semen with hot water. Hot water extraction is a simple and cost-effective process, which is advantageous because this process more effectively dissolves components of a liquid preparation that is mainly composed of water when the liquid preparation is produced using an extract of Nelumbinis Semen. The water is preferably distilled water not containing impurities, and particularly preferably triple distilled water.

The hot water extraction is preferably carried out at 80-100° C. for 1-3 hours. When the hot water extraction is carried out at less than 80° C. or for less than one hour, effective components of Nelumbinis Semen are not extracted effectively. When the hot water extraction is carried out at above 100° C. or the extraction time exceeds three hours, effective components of Nelumbinis Semen are liable to be degraded. Thus, the hot water extraction is preferably carried out within the temperature and time range.

If desired, after the hot water extraction, the resulting extract is filtered to be concentrated and freeze-dried.

The hot water extraction is preferably carried out under reflux conditions. Reflux extraction, distillation under pressure, etc. are available for the hot water extraction. The reflux extraction is particularly preferable.

The present inventors determined that the Nelumbinis Semen extract of the present invention has antidepressive activity, as follows. After the Nelumbinis Semen extract of the present invention was administered to white rats for one day or six days for evaluating chronic effects, a forced swim test was performed the next day. During forced swimming, the immobility time, when a rat ceases struggling and remains floating motionless in the water, was measured. The forced swimming itself induces depression in rats. Rats in water baths struggle first, and then stop struggling and remain floating motionless in the water. At the immobile state, rats become desperate and almost come to give up their lives. This time is considered to be the situation when depression is induced. Typically, most antidepressants significantly reduce the duration of this state.

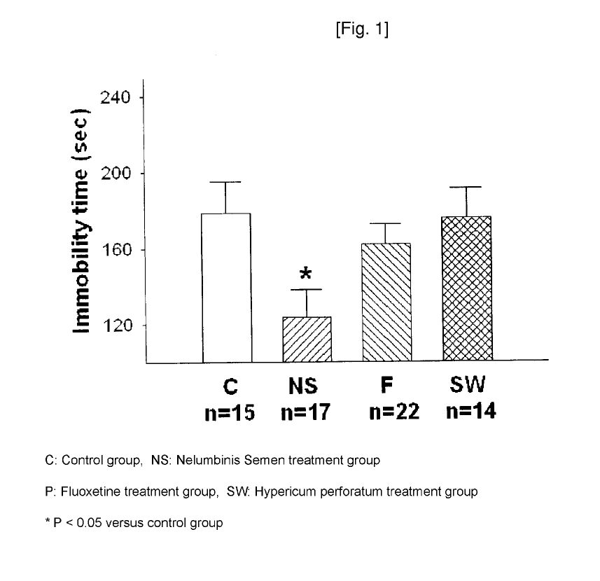

The antidepressive effects of drugs are assessed using this principle. Before being administered with drugs, experimental animals (rats) are dropped into water baths and forced to swim for 15 minutes to be trained to adapt to a suddenly changed environment without fear. When rats were administered with drugs and forced to swim, as shown in FIG. 1, the Nelumbinis Semen extract of the present invention displayed antidepressive activity. When the Nelumbinis Semen extract of the present invention and a Hypericum perforatum extract and fluoxetine as comparative groups were administered to rats for one day and compared for antidepressive effect within effective doses, only the Nelumbinis Semen extract was found to have a strong antidepressive effect by significantly reducing the immobility time of rats. Also, when the drugs were administered for six days and compared for antidepressive effect within effective doses, the Nelumbinis Semen extract was found to have the strongest antidepressive effect.

In addition, the Nelumbinis Semen extract of the present invention was tested for its antidepressive activity and for overcoming sexual dysfunction, which is a representative side effect of conventional antidepressants. This test was carried out using the aforementioned CMS model of depression in rats, being applicable to practical situations. Rats were exposed to CMS to induce depression, and were confirmed to be in a depressed state when sucrose intake decreased and the rats exhibited anhedonia (loss of interest or pleasure). Then, for the last four weeks of the test period, the rats were administered with conventional antidepressants, Prozac and a Hypericum perforatum extract, and the Nelumbinis Semen extract of the present invention. The antidepressive effects and side effects of the administered drugs were evaluated by objective comparison between test groups for behavioral changes including changes in weight, sucrose intake and physical activity in an open place. Also, a reduction in sexual behavior, which is a representative side effect of the SSRI depressants, was investigated by comparison of mating behavior between test groups according to the above drugs. As a result, the Nelumbinis Semen extract of the present invention was found to have a stronger antidepressive activity than the Hypericum perforatum extract and Prozac, used as comparative groups, while not displaying reduced sexual behavior that is a side effect of the above conventional antidepressive drugs, thereby indicating that the present Nelumbinis Semen extract does not have the side effects found upon the application of conventional antidepressive drugs.

Further, the mechanism of the antidepressive action of the Nelumbinis Semen extract of the present invention was assessed by the following biochemical method.

The therapeutic efficacy of a candidate drug was primarily examined by measuring changes in 5-HT and norepinephrine (NE) levels in a chronic CMS model by microdialysis and HPLC-ECD. Catecholamine content was measured using an HPLC system equipped with an electrochemical detector. A mobile phase containing 0.05 M monobasic sodium phosphate, 0.1 N sodium acetic acetate and 1% methanol was adjusted to pH 4.4 with a phosphate buffer for HPLC. DA was composed of a Supelcosil LC-8-DB 3- [symbol] column (150×4.6 mm, Supelco, Bellefonte, Pa.) preceded by an LC-18 guard column. As a result, the Nelumbinis Semen extract of the present invention was found, like Prozac used as a comparative group, to significantly increase the levels of the 5-HT neurotransmitter.

Secondarily, antidepressive effects were evaluated by an increase or decrease in receptor binding of a serotonin 1A receptor agonist, [³H]-8-OH-DPAT, in the rat prefrontal cortex, hippocampus and hypothalamus, which play important roles in depression, as follows. Rats were exposed to CMS for eight weeks and administered with drugs for the last four weeks of a test period. Whole brains were excised from the rats and rapidly frozen in dry ice. The rapidly frozen brains were stored at −70° C. until sectioned. The brains were cryo-sectioned into a size of 20 [symbol] at −20° C. using a cryostat microtome (Leika), and each section was attached onto a gelatin-coated slide (Superfrost/Plus, Fisher Scientific). The slides were stored at −70° C. until use for observing changes in 5-HT1A receptor levels or activity. The changes in 5-HT1A receptor were examined using a radioisotope, [³H]-8-OH-DPAT. The slides were taken out from a −70° C. freezer where the slides had been stored, and were pre-incubated in Tris-HCl buffer (170 mM Tris, 4 mM CaCl2, pH 7.6) for 30 min at room temperature. Then, the slides were incubated in Tris-HCl buffer containing 2 nM [³H]-8-OH-DPAT (Amersham) for one hour. This incubation is total binding for observing the changes in the whole 5-HT1A receptor, and non-specific binding was observed using Tris-HCl buffer containing 1 μM 5-HT (Sigma). Then, the slides were washed with Tris-HCl buffer precooled at 4° C. six times and dried with chilled air. The slides were completely dried in a desiccator for 24 hours. The completely dried slides are exposed to Hyperfilm along with a standard scale bar for five weeks. The film exposed for five weeks was developed using a developer and a fixer. For quantitative analysis of density, the film was scanned using an image analysis program, ImageQuant (Molecular dynamics). When test groups were compared with a control group for an increase or decrease in receptor binding as an indication of antidepressive action, the Nelumbinis Semen extract of the present invention was found, like the Hypericum perforatum extract used as a comparative group, to significantly increase the binding of the serotonin 1A receptor agonist, [³H]-8-OH-DPAT in all of the three brain tissues.

Thirdly, an increase in marker proteins of antidepression, which play important roles in depression, was examined in the rat hippocampus by 2-DE, as follows. Rats were exposed to CMS for eight weeks and administered with drugs for the last four weeks of a test period. The hippocampus was excised from the rats, suspended with a sonicator and centrifuged, and the supernatants were recovered. Each sample was mixed with an IEF sample buffer and loaded onto an IEF gel. Proteins were separated according to isoelectric point by isoelectric focusing (IEF) in the first dimension and according to molecular weight by SDS-PAGE at 100-200 V for about 1-2 hours in the second dimension. The gel was stained by a Gel-Code Blue staining method and evaluated for elevated or newly emerged proteins by the antidepressive substances. The elevated or newly emerged proteins by the antidepressive substances were subjected to mass spectrometry. As a result, the Nelumbinis Semen extract of the present invention was found, unlike Prozac, to significantly increase protein levels of adenylsuccinyl synthetase, cytochrome C oxidase, MAP kinase 2 and aldehyde dehydrogenase 1.

In addition, the present invention provides a pharmaceutical composition for treating depression, comprising the Nelumbinis Semen extract as an effective component.

The present pharmaceutical composition for treating depression includes the Nelumbinis Semen extract as an effective component. The pharmaceutical composition may be administered orally or parentally and may be formulated into typical pharmaceutical preparations.

That is, the Nelumbinis Semen extract of the present invention may be formulated into various pharmaceutical preparations for oral and parenteral administration upon clinical application. In the formulation, diluents or excipients may be used, which are exemplified by fillers, thickeners, binders, humectants, disintegrators and surfactants.

Examples of solid formulations for oral administration include tablets, pills, powders, granules and capsules. The solid formulations may include, in addition to the Nelumbinis Semen extract, at least one excipient selected from among starch, calcium carbonate, sucrose, lactose, gelatin, etc. Also, the solid formulations may include, in addition to a simple excipient, a lubricant such as magnesium stearate or talc.

Examples of liquid formulations for oral administration include suspensions, internal solutions, emulsions and syrups. The liquid formulations may include, in addition to commonly used simple diluents such as water and liquid paraffin, various excipients which are exemplified by humectants, sweeteners, aromatics and preservatives.

Examples of preparations for parenteral administration include sterile aqueous solutions, non-aqueous solutions, suspensions, emulsions, freeze-dried preparations and suppositories. In the formulation into non-aqueous solutions and suspensions, propylene glycol, polyethylene glycol, vegetable oils such as olive oil, and injectable esters such as ethyl oleate may be used. As a base of suppositories, witepsol, macrogol, Tween 61, cacao fat, lanolin fat, glycerol and gelatin may be used.

The unit dose, may, for example, occurs one, two, three or four times, or a half, third or quarter of an individual dose. The individual dose preferably contains the amount of an effective compound which is given in one administration and usually corresponds to a whole daily dose or a half, third or quarter of the daily dose.

In the pharmaceutical composition for treating depression, an effective amount of the Nelumbinis Semen extract ranges from 30 to 700 mg/kg, and preferably 100 to 500 mg/kg, and may be administered once to six times daily. The dosage for a specific patient may vary according to the patient's weight, age, gender, health state and diet, administration duration, administration routes, excretion rates and severity of the illness.

When the Nelumbinis Semen extract of the present invention was orally, intraperitoneally and subcutaneously administered to white rats to evaluate its toxicity, 50% lethal dose (LD50) of the Nelumbinis Semen extract upon the intraperitoneal administration was higher than 20 g/kg. This result demonstrates that the Nelumbinis Semen extract is safe.

In addition, the present invention provides a health food for treating depression, comprising the Nelumbinis Semen extract as an effective component.

In the case of using the present extract as a food, the present extract may be added as it exists or in combination with other food or food ingredients, and may be used suitably according to general methods. Mixed amounts of effective components may be suitably determined according to the intended use (prophylactic, health or therapeutic purposes). Typically, the present extract may be added in an amount of 0.01 to 1 wt %, and preferably 0.1 to 1 wt %, based on the total weight of raw materials used in preparing a food or drink. An effective amount of the present extract may be determined based on an effective amount of the pharmaceutical composition. When consumed for a long period of time for health and sanitary purposes or health control, the present extract may be used in an amount lower than the range. Also, it is apparent that the present extract can be used in an amount higher than the range because the effective component carries no safety risk.

The type of the food is not particularly limited. Examples of foods to which the present extract can be added include meats, sausages, breads, chocolates, candies, snacks, confectionary, pizza, instant noodles, other noodles, chewing gums, dairy products including ice creams, various soups, beverages, teas, drinks, alcoholic beverages and vitamin complexes, as well as traditional therapeutic preparations for use as an antianemic, a body function-strengthening agent, a skin whitening agent, and the like. In addition, the present invention may be used in various prescriptions of Chinese medical decoctions, such as Reu Do Han Shao Tang, Quing Sin Shan Yao Tang and Tai Yin Tiao Wei Tang.

### MODE FOR THE INVENTION

A better understanding of the present invention may be obtained, in conjunction with the accompanying drawings, through the following examples and test examples which are set forth to illustrate, but are not to be construed as the limit of the present invention.

### EXAMPLE 1

### Preparation of a Nelumbinis Semen Extract

500 g of Nelumbinis Semen dried powder was placed into a flask containing 1 L of triple distilled water and subjected to hot water extraction under reflux at 100° C. for one hour. The resulting solution filtered through a gauze, and the filtrate was concentrated using a vacuum filter (Eyela, Japan) and freeze-dried, thus yielding 106 g of a Nelumbinis Semen extract according to the present invention.

### TEST EXAMPLE 1

### Evaluation of Antidepressive Activity of the Nelumbinis Semen Extract by an Animal Behavioral Test

The Nelumbinis Semen extract of the present invention was orally administered to postnatal 85-95-day Sprague-Dawley male rats. A comparative group was orally administered with a Hypericum perforatum extract. Another comparative group was intraperitoneally administered with fluoxetine. Each drug was administered for one day or six days.

Before the drug administration, the white rats were dropped into a cylindrical water bath (20 cm in diameter; 40 cm water depth) and forced to swim for 15 min. The day after the drug administration for one day or six days, the rats were forced to swim for 5 min, and during this forced swimming, immobility time was measured.

As shown in FIG. 1, during the forced swimming after the one-day drug administration, the comparative groups administered with the Hypericum perforatum extract and fluoxetine showed non-significant decreases in immobility time of 1.5% and 9.3%, respectively, in comparison with a control group. In contrast, the Nelumbinis Semen extract significantly reduced the immobility time by 30.6%.

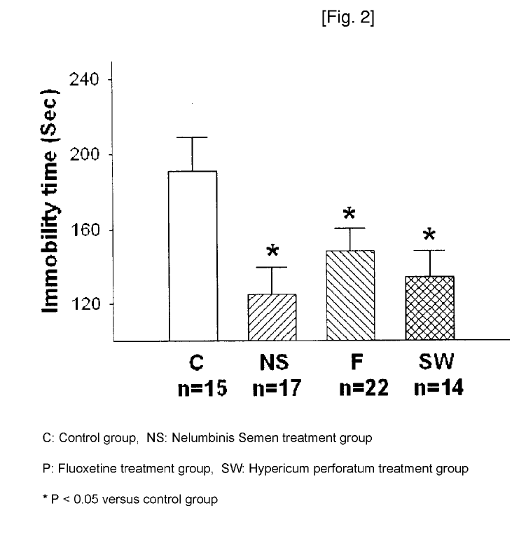

In addition, as shown FIG. 2, during the forced swimming after the six-day drug administration, the comparative groups administered with the Hypericum perforatum extract and fluoxetine showed a non-significant decrease in immobility time by 29.6% and 22.1%, respectively in comparison with a control group. In contrast, the Nelumbinis Semen extract significantly reduced the immobility time by 34.3%.

These results indicate that the Nelumbinis Semen extract of the present invention has antidepressive activity and is superior to the Hypericum perforatum extract and fluoxetine used as comparative groups in counteracting depression within an effective dose of each drug.

### TEST EXAMPLE 2

### Evaluation of Antidepressive Activity of the Nelumbinis Semen Extract by an Animal Behavioral Test in a CMS Model of Depression

Postnatal 6-week-old male Wistar white rats weighing 180-200 g were divided into test groups and a control group. The rats were first trained to experience and drink a 1% sucrose solution for 48 hrs to be adapted to sweetness. After three days, the test groups were exposed to CMS (chronic mild stress) for a total of eight weeks. The CMS procedure applied to the test groups was based on the procedure originally designed by Willner et al. (1987). The rats were sequentially exposed to a variety of mild stressors, and a series of stressors was suitably divided over one week and applied to rats according to a predetermined time schedule. This procedure was repeated for eight weeks.

The stress procedure is given in Table 1, below, which describes a weekly CMS regime (20-h food and water deprivation, prey limitation after the food and water deprivation (45 g powdered prey sprinkled), exposure to empty water bottles after the food and water deprivation, all day illumination, 30° cage tilt; soiled cage (400 ml of water spilled on bedding), presentation of intermittent 85-dB white noise, presentation of flashes of light with a stroboscope, and tripled housing). The control rats were weekly exposed only to food and water deprivation for 20 hrs before sucrose intake was measured, and were normally bred in single cages for the rest of the time with sufficient water and food supply. For the measurement of sucrose solution intake, the rats received sufficient water and food except for the water and food deprivation once pre week for the control animals and the deprivation schedule of the CMS procedure for test animals.

TABLE 1

| | | Fri | Sat | Sun | Mon | Tue | Wed | Thu |
|---|---|---|---|---|---|---|---|---|
| am. | 1 | Water/Food | Tripled | | | Fooddeprivation | | |
| | 2 | deprivation | housing | | | Soiled cage | Strobos. | Strobos. |
| | 3 | | | White | White | | | |
| | 4 | | White | noise | noise | | | |
| | 5 | | noise | | | | | |
| | 6 | | | White | noise | | White | |
| | 7 | | | | | | noise | Strobos. |
| | 8 | | | | | | | |
| | 9 | | | Strobos. | Fooddeprivation | Powered | Light | |
| | | | | | | | ON | |
| | 10 | | | | Soiled | food | TiltCage | |
| | 11 | | | | cage | | Empty | White |
| | | | | | | | bottle | |
| | 12 | | Tiltcage | | | Waterdeprivation | | noise |
| pm. | 1 | | | | | Strobos. | | |
| | 2 | Sucrose intake | | Strobos. | | | | |
| | | measurement | | | | | | |
| | 3 | | | | | | | |
| | 4 | Tripled | | | | | | Weightmeasurement |
| | 5 | housing | | | | | | |
| | 6 | | | | | | | Water/Food |
| | | | | | | | | deprivation |
| | 7 | | White | White | | | | |
| | 8 | | noise | noise | | | | |
| | 9 | | | | | Light OFF | Light | |
| | | | | | | | OFF | |
| | 10 | | | | | Whitenoise | Strobos. | |
| | 11 | | White | White | | | | |
| | 12 | | noise | noise | | | | |

<Drug Administration>

During the CMS period, when stressed rats displayed a significant decrease in sucrose intake (the core symptom of depression) compared to non-stressed rats not exposed to CMS, they were orally administered with the Nelumbinis Semen extract of the present invention. Two comparative groups were administered orally with a Hypericum perforatum extract and intraperitoneally with fluoxetine, respectively.

<Evaluation of Behavioral Changes>

(1) Sucrose Intake and Body Weight Measurement

Intake of a 1% sucrose solution was measured once every week in each of the four groups. Immediately after the 20-h water and food deprivation, rats were allowed to consume simultaneously a 1% sucrose solution and water contained in a bottle for one hour. The bottles were weighed before and after the exposure to sucrose to measure sucrose solution and water intake. Sucrose preference was expressed as a ratio of sucrose intake to total water and sucrose intake. Also, sucrose solution intake was compared between the CMS and control groups to determine whether the CMS group developed depression. In each group, sucrose intake was monitored from the beginning of CMS and for the CMS period of eight weeks. Body weight was measured before the 20-h water and food deprivation performed to measure sucrose intake every week.

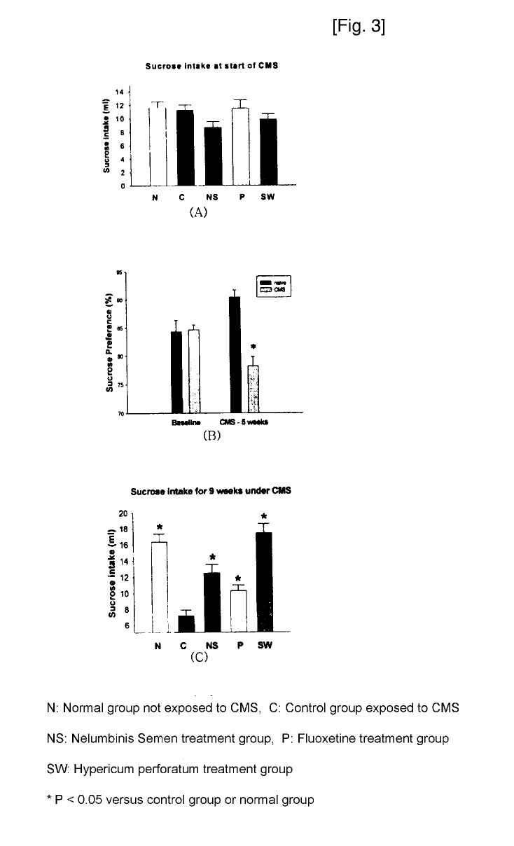

As shown in the (A) panel of FIG. 3, no change in sucrose intake was observed at the starting point of the CMS procedure. However, as shown in the (B) panel of FIG. 3, after five weeks of CMS, a CMS group significantly reduced sucrose intake while displaying a sucrose preference reduced by 16% compared to a control group not exposed to CMS. This decreased sucrose intake represents the induction of depression. Thus, drug administration was started five weeks after the beginning of CMS and carried out for the last four weeks of the 8-week CMS period. As shown in the (C) panel of FIG. 3, compared to a normal (N) group not exposed to CMS, a control group exposed to CMS significantly reduced sucrose intake by 56%, indicating that CMS induced in rats a depressive symptom, anhedonia, which is a state in which animals do not feel pleasure for sucrose consumption that would ordinarily be pleasurable. When rats were administered with the Nelumbinis Semen extract, the Hypericum perforatum extract and fluoxetine for the last four weeks of the entire CMS period, the sucrose consumption was restored to levels similar to the normal group, indicating that all administered drugs have antidepressive effects. In particular, the Nelumbinis Semen extract restored the sucrose intake to 79% of that of the normal group. These results indicate that the Nelumbinis Semen extract also has an antidepressive effect similar to that of the conventional antidepressants in the CMS animal model mimicking human depression.

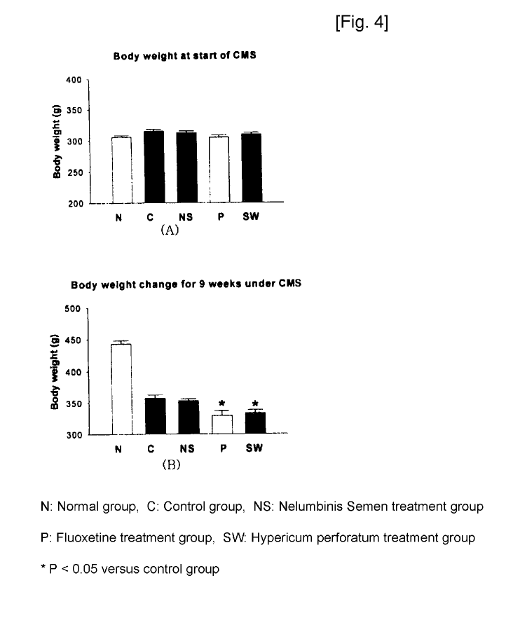

The results of body weight measurement are given in panel (A) of FIG. 4. As shown in panel (A) of FIG. 4, no difference between groups is observed at the beginning of CMS. However, after the 8-week CMS period, the control group (exposed to CMS) showed great weight loss in comparison with the normal group. This weight loss is a common symptom of depression induced by CMS. When rats were administered with drugs for the last four weeks of the CMS period, only a Nelumbinis Semen extract treatment group showed body weight similar to that of the control group but did not restore body weight to that of the normal group. In contrast, as shown in panel (B) of FIG. 4, two groups administered with the Hypericum perforatum extract and fluoxetine, respectively, displayed greater weight loss than the control group, indicating that the conventional antidepressive drugs cause severe side effects.

These results demonstrate that the Nelumbinis Semen extract reverses the weight loss, a severe side effect or toxicity of conventional antidepressants, and is thus a safe drug without the toxicity of conventional antidepressants.

(2) Open Field Activity

In order to evaluate the activity difference CMS exposure and drug administration, several behaviors of rats were observed in an open field after the 9-week measurement of sucrose solution intake was completely finished. The open field consisted of a wooden box of 75×75×30 cm, the floor of which was divided by width and length lines into 15 cm squares. The open field was connected to a small box of 15×15×15 cm, which was used as a start box where experimental animals waited to enter the open field. A vertical sliding door was installed between the start box and the open field. White rats were placed in the start box, and, after 30 sec, the sliding door was open. The time to leave the start box toward the open field, which was expressed as start latency, was recorded. The start latency time means the time ranging from when the door was opened to when the rat tails completely exited the start box. Immediately after the start latency, open field behaviors of rats were observed. The following behaviors were recorded for a total of 10 min: locomotion, rearing, grooming and defecation. Grooming behavior was recorded as the total time spent grooming in 10 min, and the remaining behaviors were all recorded as frequencies. Locomotor activity was recorded by counting the number of squares each white rat crossed on the floor divided into 25 15×15-cm squares using a computer tracking system. Rearing was recorded as the total number of times each rat displayed exploratory behavior while standing on its hind paws during 10 min. Grooming was recorded as the number of times each rat stroked its head or combed its fur with its forepaws during 10 min. After the 10-min observation, the white rats were removed from the open field, and defecation frequency was recorded by counting fecal boluses on the floor of the open field.

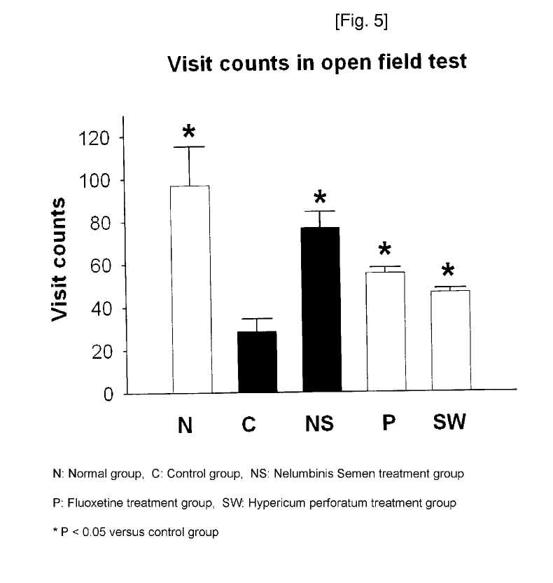

After the drug administration for the last four weeks of the 8-week CMS period, the total number of chambers which each animal visited (visit counts) was recorded by an open field test. As shown in FIG. 5, compared to a control group (exposed to CMS, visited an average of 30 places in 10 min), a Nelumbinis Semen treatment group displayed a significant increase in visit counts (visited an average of 75 places in 10 min, P<0.05). These visit counts are greater than those of the representative conventional antidepressants, Prozac (visited an average of 60 places in 10 min, P<0.05) and natural St. John's wort (visited an average of 50 places in 10 min, P<0.05), demonstrating that the Nelumbinis Semen extract of the present invention has a stronger antidepressive effect. Also, these results indicate that the Nelumbinis Semen administration reverses the rat activity reduced due to the CMS-induced depression.

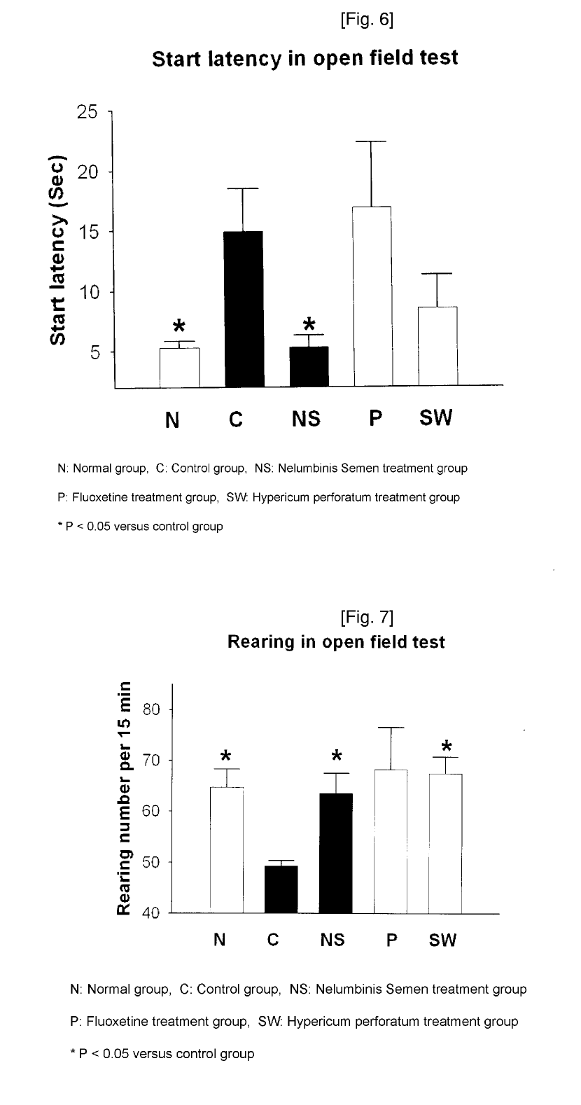

The start latency time was measured as follows. After the drug administration for the last four weeks of the 8-week CMS procedure, rats were subjected to an open field test, and the time to leave the start box toward the open field when the sliding door was recorded. As shown in FIG. 6, compared to the control group, only the Nelumbinis Semen treatment group displayed a significant decrease (P<0.05) in start latency. The Nelumbinis Semen extract was found to have a stronger antidepressive effect than conventional antidepressants, Prozac and natural St. John's wort. This Nelumbinis Semen administration resulted in shortened start latency. These results indicate that the CMS-induced reduction in curiosity and the will to live was reversed by treatment with Nelumbinis Semen extract.

Rearing behavior was also recorded after the drug administration for the last four weeks of the 8-week CMS procedure. Rats were subjected to an open field test, and rearing frequency was recorded. As shown in FIG. 7, compared to the control group, the Nelumbinis Semen treatment group displayed a significant decrease (P<0.05) in rearing frequency. The Nelumbinis Semen extract was found to have a stronger antidepressive effect than conventional antidepressants, Prozac. These results indicate that the Nelumbinis Semen administration restored the exploratory behavior of rats to a new environment. That is, these results indicate that the CMS-induced reduction in the curiosity and exploratory behavior to neighboring environments was reversed by the treatment with the Nelumbinis Semen extract.

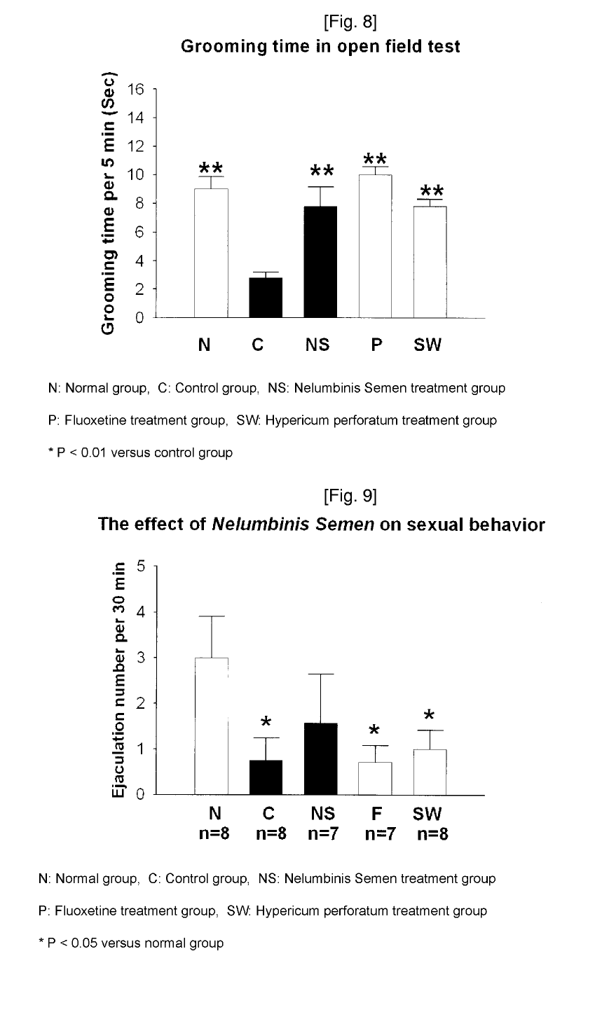

Grooming behavior was recorded after the drug administration for the last four weeks of the 8-week CMS procedure. Rats were subjected to an open field test, and the time spent self-grooming was measured. As shown in FIG. 8, compared to the control group, the Nelumbinis Semen treatment group displayed a significant increase (P<0.05) in grooming time. This antidepressive effect was similar to that of a conventional antidepressant, the St. John's wort plant. These results indicate that the Nelumbinis Semen administration restored the interest of the rats in themselves. That is, these results indicate that the CMS-induced reduction in self-interest was reversed by treatment with the Nelumbinis Semen extract.

(3) Sexual Behavior

A decrease in sucrose consumption (the core symptom of depression) in rats was confirmed during the 8-week CMS period, and antidepressants were administered for the last four weeks. Then, mating behavior of rats was observed. This test was carried out a dark and quiet room using a 16×16-inch transparent acrylic box.

Mature female rats were ovariectomized, and, to induce sexual receptivity, were administered with 20 μg of estradiol benzoate 48 hrs before the test and 1 mg of progesterone 5 hrs before the test. Male rats were singly housed in the same box for 30 min to adapt to a new environment. Then, the female rats were placed into the box, and sexual behavior was observed for 30 min. When female rats have sexual receptivity, that is, are estrous, they allow male rats to mount and show lordosis. Sexual behaviors of male rats were divided into mounting, intromission and ejaculation, and frequency of each behavior was recorded. The number of ejaculation (higher frequency generally means higher sexual activity) was recorded for 30 min. The time taken after ejaculation before resumption of sexual activity was called “post-ejaculation interval (PEI)”. A decrease in PEI generally means that male rats have increased sexual activity.

The ovariectomized female rats, administered with estradiol and progesterone, and male rats were introduced into the acrylic mating arena, and ejaculation frequency was recorded for 30 min. As shown in FIG. 9, in animals (male rats) administered with fluoxetine and St. John's wort, respectively, the sexual behavior was remarkably reduced compared to normal animals. There was no statistic difference in sexual behavior between animals administered with the Nelumbinis Semen extract and control animals. These results indicate that the sexual behavior suppressed by CMS was reversed by Nelumbinis Semen administration.

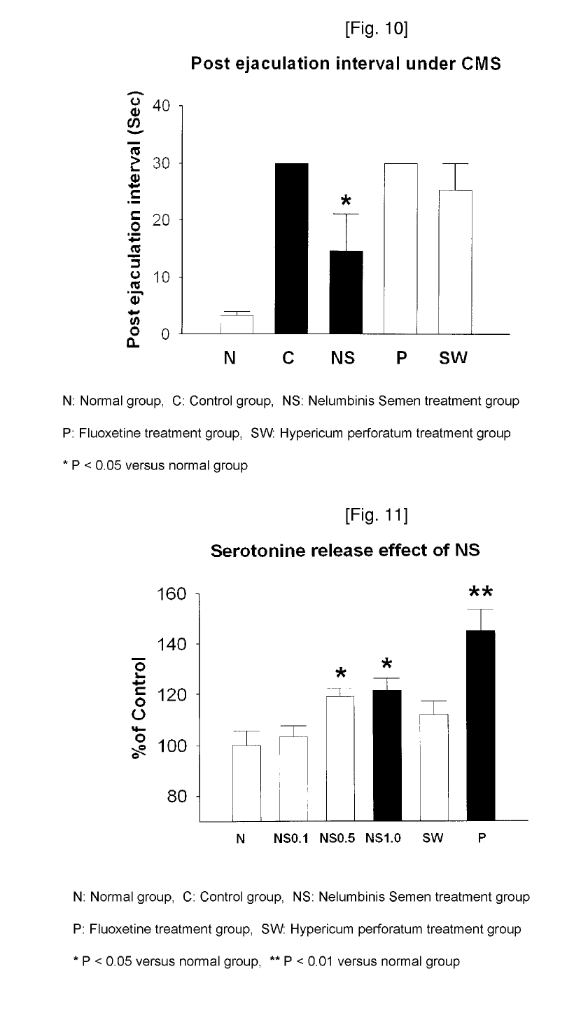

The ovariectomized female rats, administered with estradiol and progesterone, and male rats were introduced into the acrylic mating arena, and post-ejaculation interval (PEI) was recorded for 30 min. As shown in FIG. 10, no significant difference was found between animals (male rats) administered with fluoxetine and St. John's wort, respectively, and control animals (C) exposed to CMS. In contrast, a statistical difference in sexual behavior was found between animals administered with the Nelumbinis Semen extract and control animals. That is, PEI was significantly reduced in animals administered with fluoxetine and St. John's wort. These results indicate that the sexual behavior suppressed by CMS is reversed by Nelumbinis Semen administration.

### TEST EXAMPLE 3

### Identification of the Mechanism of Antidepressive Action of the Nelumbinis Semen Extract

The Nelumbinis Semen extract was assessed for the antidepressive efficacy by measuring changes in 5-HT and norepinephrine (NE) levels in normal animals and a chronic CMS model by microdialysis and HPLC-ECD. This test is based on examining the direct effect of antidepressants on the increase or decrease in neurotransmitter levels, which is the core action of antidepressants. The Nelumbinis Semen extract was administered to normal animals (not exposed to CMS), and its direct effect on neurotransmitter levels was examined. As shown in FIG. 11, when normal animals were administered with the Nelumbinis Semen extract (100 mg/kg, 500 mg/kg and 1000 mg/kg, p.o.), St. John's wort extract (500 mg/kg, p.o.) and fluoxetine (10 mg/kg, i.p.), respectively, the Nelumbinis Semen extract significantly increased 5-HT levels at doses of 500 and 1000 mg/kg, and fluoxetine significantly increased 5-HT levels at a dose of 10 mg/kg. The St. John's wort extract, administered at a dose of 500 mg/kg, increased 5-HT levels but did not show a statistic significance. The increased serotonin levels by the Nelumbinis Semen treatment in normal animals, which were similar to those by the fluoxetine treatment indicate that the Nelumbinis Semen extract has antidepressive efficacy.

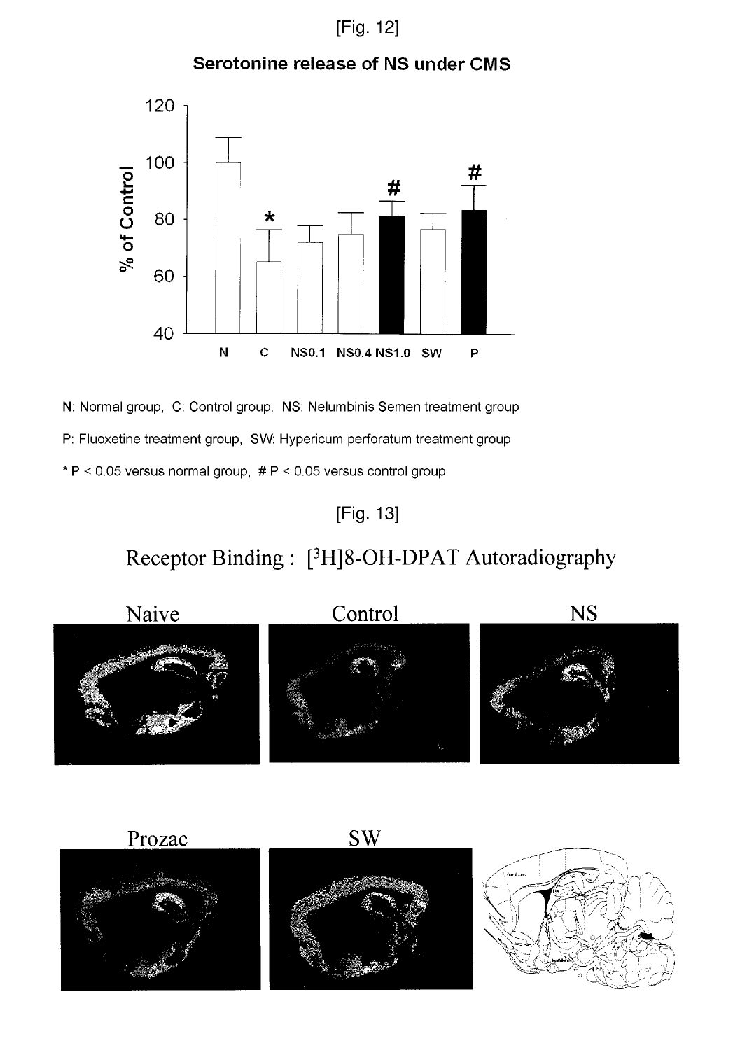

The antidepressive effect of the Nelumbinis Semen extract was also examined in a CMS model of depression. Rats were sequentially exposed to a variety of mild stressors for 8 weeks. The reversal of CMS-induced decreased 5-HT levels was investigated in the hippocampus of the rats by microdialysis and HPLC-ECD. As shown in FIG. 12, after the 8-week CMS period, control rats (exposed to CMS but administered with no drug) displayed a significant decrease in 5-HT levels in the hippocampus in comparison with normal rats (not exposed to CMS). When rats were administered with the Nelumbinis Semen extract (1000 mg/kg, p.o.) during the CMS exposure, a significant increase (P<0.05) in 5-HT levels was found in comparison with the control rats. Compared to the control group, another group administered with fluoxetine (10 mg/kg, i.p.) showed a significant increase (P<0.05) in 5-HT levels. No significant difference was found between animals administered with a St. John's wort extract (500 mg/kg, p.o.) and the control animals. These results strongly indicate that the Nelumbinis Semen extract has antidepressive effect in a CMS-induced animal model of depression.

The changes in 5-HT1A receptor levels or activity were examined using a radioisotope, [³H]-8-OH-DPAT. Reduced levels or inactivation of 5-HT1A receptor occur in the prefrontal cortex, hippocampus and hypothalamus of depressed patients. The antidepressive drug administration returns the decreased levels of the receptor to normal levels or converts an inactive form of the receptor into a normal active form, leading to an improvement in neurotrophic action, neuronal survival, etc. This normalization of 5-HT1A receptor by antidepressant administration is effective in treating depression and depression-associated neurodegeneration. Based on this fact, an increase in 5-HT1A receptor levels can be assessed by a receptor binding assay using a 5-HT1A receptor antagonist. When an increase in 5-HT1A receptor levels is observed upon Nelumbinis Semen administration in comparison with a control group exposed to CMS, this is considered an in vivo indication of an antidepressive effect at the molecular level. After drugs were administered for the last four weeks of the 8-week CMS period, an increase in 5-HT1A receptor levels, as an in vivo indication of antidepressive effect, was found in animals administered with the Nelumbinis Semen extract.

As shown in FIG. 13, a receptor binding assay using a 5-HT1A receptor antagonist resulted in the finding that, compared to a control group exposed to CMS, a Nelumbinis Semen treatment group (NS group) displayed an increase in 5-HT1A receptor levels, similar to that of a Prozac treatment group, in the rat hippocampus and hypothalamus (binding intensity: Red>yellow>green). That is, the Nelumbinis Semen administration resulted in increased 5-HT1A receptor levels, a representative indication of an antidepressive effect.

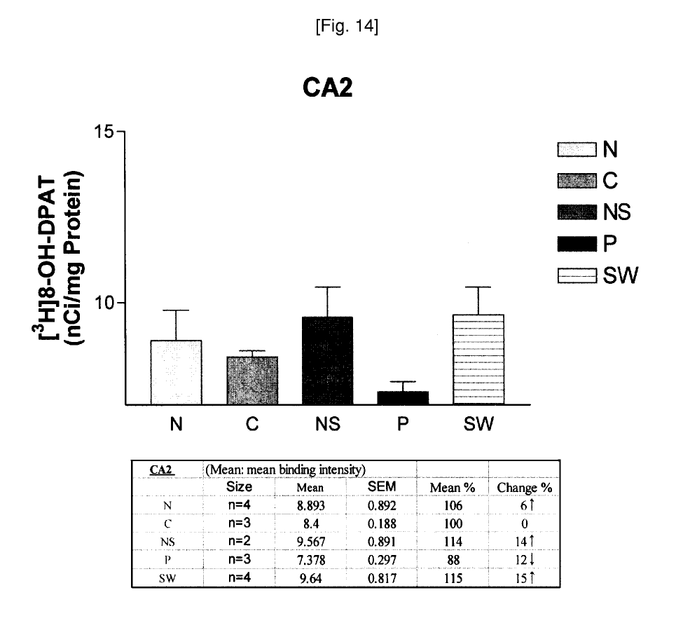

The antidepressive effect of Nelumbinis Semen was evaluated by investigating the increase in 5-HT1A receptor levels, an in vivo indication of an antidepressive effect, in rats exposed to CMS for eight weeks and administered with the Nelumbinis Semen extract for the last four weeks of the CMS period. As shown in FIG. 14, a receptor binding assay using a 5-HT1A receptor antagonist resulted in the finding that, compared to a control group, a Nelumbinis Semen treatment group (NS group) displayed an increase in 5-HT1A receptor levels, similar to that of a St. John's wort treatment group (SW group), in the CA2 region of the rat hippocampus (14% and 15%, respectively). In contrast, a Prozac treatment group (P group) displayed a decrease in 5-HT1A receptor levels. These results indicate that Nelumbinis Semen has a different mechanism of antidepressive action. Namely, the Nelumbinis Semen administration resulted in the increased 5-HT1A receptor levels, a representative indication of an antidepressive effect. The Nelumbinis Semen administration increased 5-HT1A receptor levels in the CA2 region of the hippocampus which is essentially required for memory storage and long term memory formation. The increased 5-HT1A receptor levels means the reversal of an CMS-induced impairment in CA2 of the hippocampus. However, since Prozac exhibited a reversal effect, Prozac application requires that special attention be paid to toxicity and side effects.

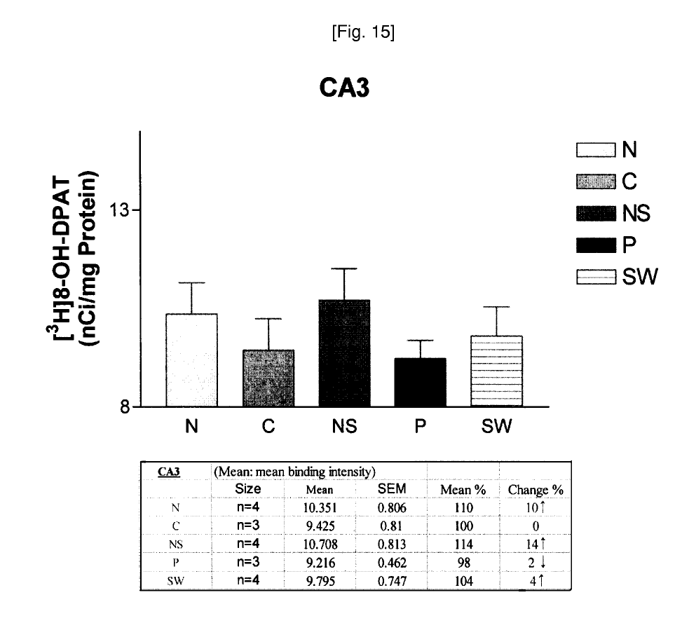

As shown in FIG. 15, a receptor binding assay using a 5-HT1A receptor antagonist resulted in the finding that, compared to a control group, a Nelumbinis Semen treatment group (NS group) displayed a higher increase in 5-HT1A receptor levels (increased by 14%) than other drugs, in the CA3 region of the rat hippocampus. In contrast, a Prozac treatment group (P group) displayed a decrease of 2% in 5-HT1A receptor levels. These results indicate that Nelumbinis Semen has a different mechanism of antidepressive action. Namely, the Nelumbinis Semen administration resulted in the increased 5-HT1A receptor levels, a representative indication of an antidepressive effect. The Nelumbinis Semen administration increased 5-HT1A receptor levels in the CA3 region of the hippocampus which is essentially required for memory storage and long term memory formation. The increased 5-HT1A receptor levels mean the reversal of CMS-induced impairment in CA3 of the hippocampus, and are expected to lead to the reversal of decreased memory and learning capacity. However, since Prozac exhibited a reversal effect, Prozac application requires that special attention to be paide to toxicity and side effects.

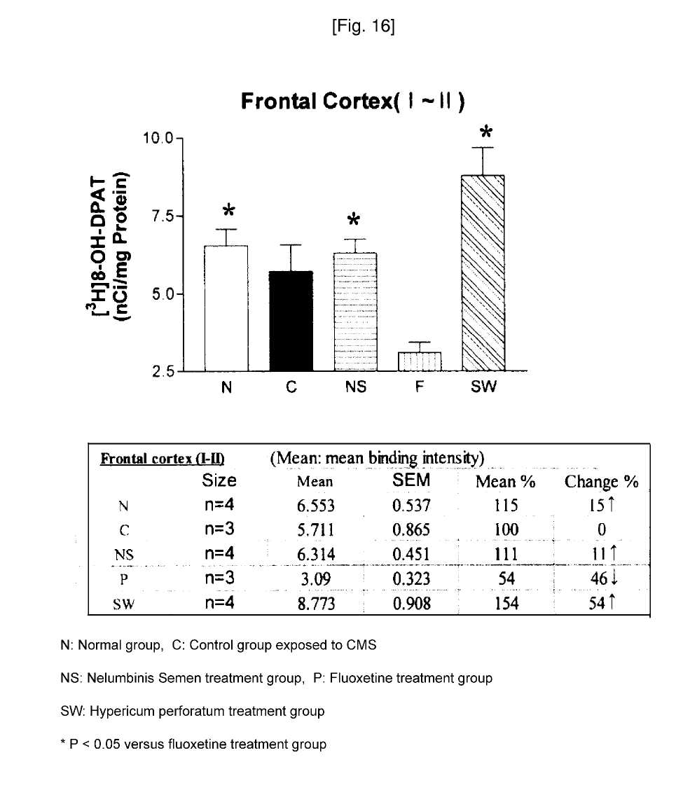

As shown in FIG. 16, a receptor binding assay using a 5-HT1A receptor antagonist resulted in the finding that, compared to a control group, a Nelumbinis Semen treatment group (NS group) displayed an increase in 5-HT1A receptor levels (increased by 11%) in layer I-II of the rat cerebral frontal cortex. In contrast, a Prozac treatment group (P group) displayed a significant decrease (P<0.05) of 46% compared to the NS group. These results indicate that Nelumbinis Semen has a different mechanism of antidepressive action. Namely, the Nelumbinis Semen administration resulted in the increased 5-HT1A receptor levels, a representative indication of an antidepressive effect. The Nelumbinis Semen administration increased 5-HT1A receptor levels in layer I-II of the cerebral frontal cortex, which participates in recognition capacity. The increased 5-HT1A receptor levels mean the reversal of CMS-induced impairment in layer I-II of the frontal cortex, and are expected to lead to the reversal of decreased recognition capacity, induced by depression. However, since Prozac exhibited a reversal effect, Prozac application requires that special attention be paid to toxicity and side effects.

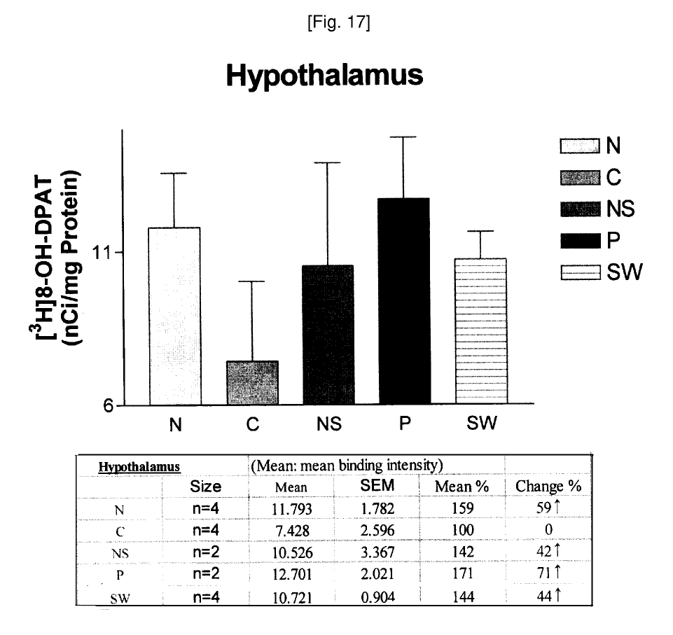

As shown in FIG. 17, a receptor binding assay using a 5-HT1A receptor antagonist resulted in the finding that, compared to a control group, a Nelumbinis Semen treatment group (NS group) displayed an increase in 5-HT1A receptor levels, similar to that of a St. John's wort treatment group (SW group), in the rat hypothalamus (42% and 44%, respectively). Namely, the Nelumbinis Semen administration resulted in increased 5-HT1A receptor levels, a representative indication of an antidepressive effect. The Nelumbinis Semen administration increased 5-HT1A receptor levels in the hypothalamus which most sensitively responds to chronic stressors and is most damaged. The increased 5-HT1A receptor levels mean the reversal of CMS-induced impairment in the hypothalamus.

Administration of antidepressants was reported to typically increase or activate a serotonin receptor by increasing serotonin levels, leading to the activation of adenyl cyclase that is downstream of the serotonin receptor to increase cAMP levels and then an activation of PKA, and eventually increases in protein levels of CREB, BDNF and its receptor trkB and ERK (MAP kinase). These proteins were reported to amplify the expression of genetic factors associated with cell growth or cell survival, so that they prevent neuronal cell damage due to depression and increase communication and plasticity between nerve cells. In order to identify the mechanism of antidepressive action of Nelumbinis Semen, in rats administered with the Nelumbinis Semen extract, an increase or decrease in expression of such antidepressive marker proteins or directly or indirectly related factors was assessed by a 2-DE system. Protein samples from the rat hippocampus were loaded onto an IEF gel. Proteins were separated according to isoelectric point by isoelectric focusing (IEF) in the first dimension and according to molecular weight by SDS-PAGE in the second dimension. Proteins increased or decreased by the Nelumbinis Semen administration were analyzed by MALDI-TOF.

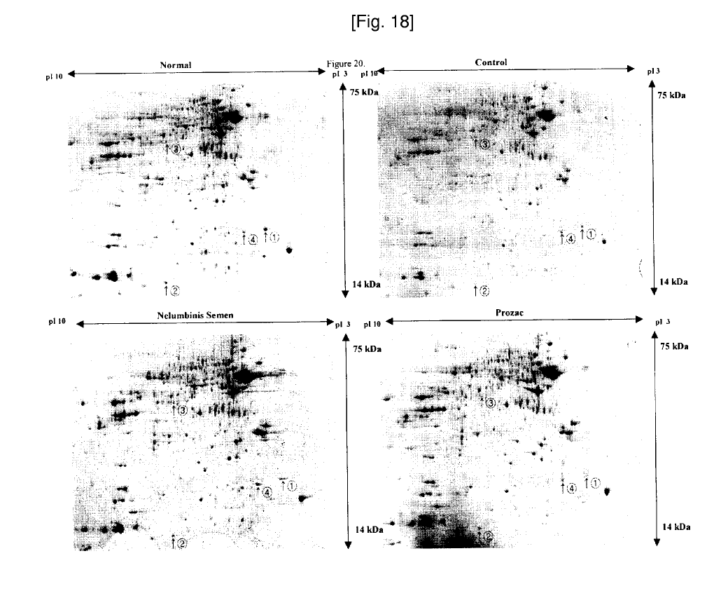

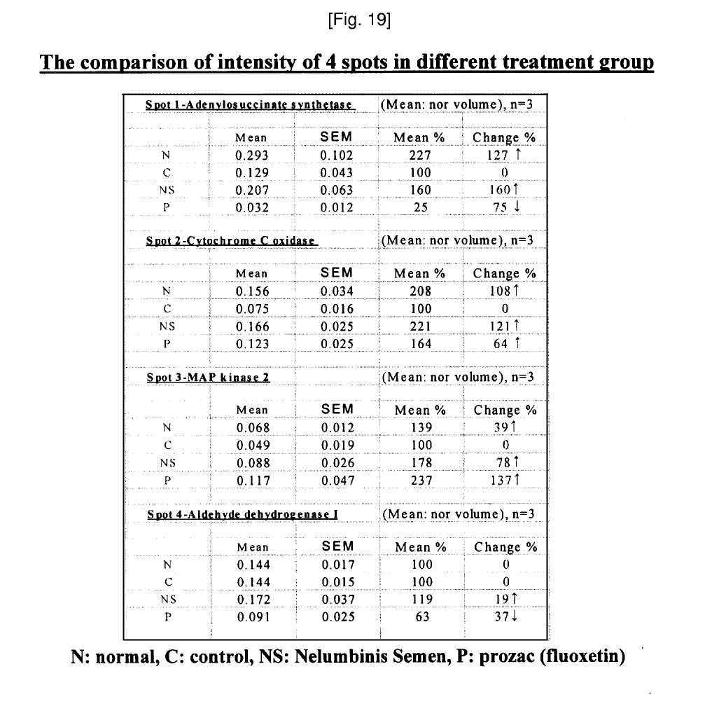

As shown in FIGS. 18 and 19, compared to a control group (exposed to CMS), a Nelumbinis Semen treatment group displayed four increased protein spots. The intensity of these spots was much stronger compared to a normal group as well as the control group. In contrast to the Nelumbinis Semen treatment, Prozac treatment resulted in a decrease in spots 1 and 4. The four increased protein spots were considered as in vivo indications specific for Nelumbinis Semen. The four proteins were all found to be associated with in vivo antidepressive markers, which are downstream of serotonin receptor activated by increased serotonin levels.

Spot 1: Adenylosuccinate Synthetase

Adenylosuccinate synthetase participates in AMP synthesis. The cAMP signal transduction mechanism in the hypothalamus and frontal cortex is associated with pathological states of depression. This enzyme was reported to be activated by growth factor. Thus, the activation of 5-HT receptor by increased serotonin levels and resulting increased growth factor expression may bring about increased expression and activation of the enzyme. In this case, an increase in cAMP levels is expected to occur very slowly. The increased cAMP levels, which lasted a long period of time and were induced by Nelumbinis Semen, indicate that drug addiction and other side effects, such as withdrawal symptoms caused by stopping of drug administration and sexual dysfunction, can be solved. In contrast, Prozac was found to inhibit the activity of the enzyme, indicating that Prozac increases cAMP levels not by an indirect pathway via the activation of the enzyme but by another pathway. That is, Prozac is believed to rapidly increase cAMP levels by a direct pathway and very rapidly change physiological systems, thereby causing drug addiction and other side effects, such as withdrawal symptoms caused by stopping of drug administration and sexual dysfunction.

Spot 2: Cytochrome C Oxidase Polypeptide VIa-Liver, Mitochondrial Precursor

Cytochrome C oxidase is a major enzyme of ATP synthesis and participates in proton transfer in the mitochondrial inner membrane. Levels of the enzyme are proportional to neuronal activity or ATP levels. A decrease in neuronal activity or ATP levels is closely related to reduced synaptic transmission of nerve signals, and depression is featured by decreased transmission of nerve signals. Thus, an increase in cytochrome C oxidase polypeptide VIa-liver, mitochondrial precursor levels may reduce depression.

Spot 3: Mitogen-Activated Protein Kinase 1 (Extracellular Signal-Regulated Kinase 2, ERK-2)

ERK-2 is known to activate nerve growth factor. The 5-Hydroxytryptamine (5-HT, serotonin) levels increased by antidepressant administration bring about the activation of serotonin receptor in the hippocampus, cause the growth of new synaptic connections, and increase the activity of MAPK that is downstream of the serotonin receptor. MAPK plays critical roles in gene expression, synaptic plasticity, receptor function and neuronal activity. Also, this enzyme has been reported to induce the synthesis of growth factors required for neuronal cell survival by activating transcription factors such as CREB. Many depressed patients displayed decreased signal transmission in synaptic connections due to decreased 5-HT levels. Thus, depression may be reduced by an increase in MAPK levels.

Spot 4: Aldehyde Dehydrogenase (ALDH)

ALDH is a key enzyme in fructose, acetaldehyde and oxalate metabolism and represents a major detoxification system for reactive carbonyls and aldehydes. According to a report, ALDH levels were reduced by 50% or more in the brain of patients with Down Syndrome compared to a control. The decreased ALDH levels result in the incomplete degradation of neurotransmitters, which can lead to the formation of plaques and tangles, thereby disturbing the nerve signal transmission system in the brain. Also, ALDH deficiency is closely related with a non-functional mutant of 5-HT receptor. Thus, the activation of ALDH by Nelumbinis Semen may be related with the protection of 5-HT receptor, which may lead to more activation of 5-HT receptor, resulting in the achievement of a much stronger antidepressive effect. In contrast, the ALDH levels reduced by Prozac may accelerate the reduction of another neurotransmitter of antidepressive action, norepinephrine (NE), and more occurrence of non-functional 5-HT receptor mutants. Thus, Prozac may cause toxicity and side effects.

### TEST EXAMPLE 4

### Evaluation of Acute Toxicity of the Nelumbinis Semen Extract Upon Oral Administration to Rats

An acute toxicity test was carried out using 6-week specific pathogen-free (SPF) SD rats. The Nelumbinis Semen extract of the present invention was suspended in a 0.5% methylcellulose solution and orally administered to groups each consisting of five rats, in a single dose of 5 g/kg, 10 g/kg and 20 g/kg. After administration of the extract, death, clinical symptoms and weight change were observed, and a hematological test and hematobiochemical analysis were performed. Upon autopsy, abnormality of abdominal organs and chest organs was visually observed.

As a result, all rats administered with the extract exhibited no particular clinical symptoms, no death, no change in weight and no toxicity upon the hematological assay, hematobiochemical analysis and autopsy. As a result, the Nelumbinis Semen extract of the present invention exhibited no toxicity even at a dose of 10 g/kg in all rats, and thus had a 50% lethal dose (LD50) higher than 20 g/kg upon oral administration. This result demonstrates that the Nelumbinis Semen extract is safe.

### FORMULATION EXAMPLE 1

### Preparation of Soft Capsules

Soft capsules were prepared according to a soft capsule preparation method described in General Rules for Preparation in a guidebook, Korean Pharmacopoeia, using 100.0 mg per capsule of the Nelumbinis Semen extract prepared in Example 1, 175.0 mg of soybean oil, 45.0 mg of cera flava, 127.5 mg of hydrogenated palm oil, 21.0 mg of soybean phospholipids, 212.0 mg of gelatin, 50.0 mg of glycerin (gravity: 1.24), 76.0 mg of di-sorbitol, 0.54 mg of methyl-paraoxybenzoate, 0.90 mg of propyl-paraoxybenzoate, 0.56 mg of methylvanillin, and a proper amount of yellow no. 203.

### FORMULATION EXAMPLE 2

### Preparation of Tablets

100.0 mg of the Nelumbinis Semen extract prepared in Example 1, 90.0 mg of corn starch, 175.0 mg of lactose, 15.0 mg of L-hydroxypropylcellulose, 5.0 mg of polyvinylpyrolidone 90 and a proper amount of ethanol were homogeneously mixed, granulated by wet granulation, mixed with 1.8 mg of magnesium stearic acid, and forced into 400 mg tablets.

### FORMULATION EXAMPLE 3

### Preparation of Capsules

100.0 mg of the Nelumbinis Semen extract prepared in Example 1, 83.2 mg of corn starch, 175.0 mg of lactose and 1.8 mg of magnesium stearic acid were homogeneously mixed, and filled into capsule shells at 360 mg per capsule.

### FORMULATION EXAMPLE 4

### Preparation of Food and Beverage

The present inventors prepared food and a beverage comprising the Nelumbinis Semen extract as an effective component, as follows.

<4-1> Preparation of Chewing Gum

Chewing gum was prepared according to a general method using 0.24-0.64% of the Nelumbinis Semen extract prepared in Example 1, 20% of gum base, 76.36-76.76% of sugar, 1% of a fruit aromatic and 2% of water.

<4-2> Preparation of Ice Cream

Ice cream was prepared according to a general method using 0.24-0.64% of the Nelumbinis Semen extract prepared in Example 1, 10.0% of milk fat, 10.8% of SNF (Solids Not Fat), 12.0% of sugar, 3.0% of starch syrup, 0.5% of an emulsion stabilizer (span), 0.15% of an aromatic (strawberry) and 63.31-62.91% of water.

<4-3> Preparation of Beverage

A beverage was prepared according to a general method using 0.48-1.28 mg of the Nelumbinis Semen extract prepared in Example 1, 522 mg of honey, 5 mg of thioctic acid amide, 10 mg of nicotinic acid amide, 3 mg of riboflavin hydrochloride sodium, 2 mg of pyridoxine hydrochloride, 30 mg of inositol, 50 mg of orotic acid and 200 ml of water.

<4-4> Preparation of Sausage

Sausage was prepared according to a general method using 0.24-0.64% of the Nelumbinis Semen extract prepared in Example 1, 63.6% of pork, 27.5% of chicken, 3.5% starch, 1.7% of soybean proteins, 1.62% of edible salt, 0.5% of glucose and 0.94-1.34% of another additive (glycerin).

Practical and presently preferred embodiments of the present invention are illustrative as shown in the foregoing Examples. However, those skilled in the art will appreciate that various modifications, additions and substitutions are possible, without departing from the scope and spirit of the invention as disclosed in the accompanying claims.

1. A Nelumbinis Semen extract having antidepressive activity, which is prepared by extracting Nelumbinis Semen with hot water.

2. The Nelumbinis Semen extract having antidepressive activity according to claim 1, wherein the hot water extraction is carried out at 80-100° C. for 1-3 hours.

3. The Nelumbinis Semen extract having antidepressive activity according to claim I, which is prepared by a method further comprising, after the hot water extraction, filtering a resulting extract to be concentrated and freeze-drying a resulting concentrate.

4. The Nelumbinis Semen extract having antidepressive activity according to claim 1, wherein the hot water extraction is carried out under reflux.

5. A pharmaceutical composition for treating depression, comprising the Nelumbinis Semen extract prepared according to claim 1 as an effective component.

6. A health food for treating depression, comprising the Nelumbinis Semen extract prepared according to claim 1 as an effective component.

7. A pharmaceutical composition for treating depression, comprising the Nelumbinis Semen extract prepared according to claim 2 as an effective component.

8. A pharmaceutical composition for treating depression, comprising the Nelumbinis Semen extract prepared according to claim 3 as an effective component.

9. A pharmaceutical composition for treating depression, comprising the Nelumbinis Semen extract prepared according to claim 4 as an effective component.

10. A health food for treating depression, comprising the Nelumbinis Semen extract prepared according to claim 2 as an effective component.

11. A health food for treating depression, comprising the Nelumbinis Semen extract prepared according to claim 3 as an effective component.

12. A health food for treating depression, comprising the Nelumbinis Semen extract prepared according to claim 4 as an effective component.
